(19) United States
(12) Patent Application Publication
(10) Pub. No.: US 2022/0128541 A1
(43) Pub. Date: Apr. 28, 2022

(54) USE OF PLASMA MEMBRANE PARTICLES, LIPOSOMES, AND EXOSOMES TO ASSAY IMMUNE CELL POTENCY

(71) Applicant: RESEARCH INSTITUTE AT NATIONWIDE CHILDREN'S HOSPITAL, Columbus, OH (US)

(72) Inventors: Dean Anthony LEE, Canal Winchester, OH (US); Aarohi THAKKAR, Columbus, OH (US); Mark HALL, Columbus, OH (US); Jennifer MUSZYNSKI, Columbus, OH (US)

(21) Appl. No.: 17/431,270

(22) PCT Filed: Feb. 14, 2020
(86) PCT No.: PCT/US2020/018384

§ 371 Date: Aug. 16, 2021

Related U.S. Application Data
(60) Provisional application No. 62/805,359, filed on Feb. 14, 2019.

(51) Int. Cl. G01N 33/50; G01N 33/92; A61K 35/15; A61K 35/17; G01N 33/68; C12N 5/0783; A61P 35/00; A61K 38/17

(52) U.S. Cl. CPC G01N 33/505; G01N 33/92; A61K 35/15; A61K 35/17; A61K 38/1774; C12N 5/0646; A61P 35/00; G01N 33/5041; G01N 33/6863

(57) ABSTRACT

A method of determining the potency of an immune cell is described. The method includes the steps of contacting an immune cell with an effective amount of a cell exosome and detecting the amount of a cytokine produced by the immune cell. Kits for assaying immune cell potency are also described. Potency assays are important for satisfying the FDA requirements for new biological agents, such as immunotherapeutic cells. Methods of using potent immune cells as an immunotherapeutic treatment are described.

### RELATED APPLICATIONS

This application claims the benefit of U.S. Provisional Application No. 62/805,359, filed Feb. 14, 2019, which is incorporated herein by reference in its entirety.

### FIELD

This invention relates to immunotherapy, and more particularly to testing effector function of immune cells.

### BACKGROUND

Immunotherapy is the treatment of disease by activating or suppressing the immune system. Cells derived from the immune system may be used to improve immune functionality and characteristics. In recent years, immunotherapy has become of great interest to researchers, clinicians and pharmaceutical companies, particularly in its promise to treat various forms of cancer. Immunomodulatory regimens often have fewer side effects than existing drugs, including less potential for creating resistance when treating microbial disease.

Conventional cancer treatments focus on killing or removing cancer cells with chemotherapy, surgery, and/or radiation. However, the field of therapeutic immune cells is growing rapidly, and can be used in conjunction with or, in some cases, in place of conventional treatments to treat, prevent, or delay the onset of a cancer. Immune effector cells such as lymphocytes, macrophages, dendritic cells, natural killer cells (NK Cell), cytotoxic T lymphocytes (CTL), etc., naturally work together to defend the body against cancer by targeting abnormal antigens expressed on the surface of tumor cells. Recent cancer treatment developments have focused on directing the patient's immune system to attack and destroy tumors. A variety of strategies are in use or are undergoing research and testing.

Adoptive cell transfer (ACT) is the transfer of cells into a patient, and has shown promise against lung, melanoma, and other cancers. The cells may have originated from the patient (autologous) or from another individual (allogenic). Allogeneic therapies involve cells isolated and expanded from a donor separate from the patient receiving the cells. Alternatively, adoptive cell transfer can be used to cultivate and expand autologous, extracted cells in vitro for later transfusion. For example, autologous immune enhancement therapy involves the extraction of a subject's own peripheral blood-derived natural killer cells, cytotoxic T lymphocytes, epithelial cells and other relevant immune cells, the expansion of these cells in vitro, and then the reinfusion of these cells into the subject's body.

In some therapies, cells (for example, T cells) are genetically modified and expanded in vitro before being returned to the same patient. Chimeric antigen receptor T cell therapy (CAR-T) involves harvesting T cells from a subject and then infecting the T cells with a retrovirus that contains a copy of a T cell receptor (TCR) gene. The TCR gene is specialized to recognize tumor antigens (for example, a chimeric antigen receptor, or CAR). The virus integrates the receptor into the T cells' genome. The cells are expanded non-specifically and/or stimulated. The cells are then reinfused and produce an immune response against the tumor cells.

With the approval of first CAR-T therapy, and multiple commercial companies involved in multiple clinical trials, this field has exploded commercially and has shown a promising future for immunotherapies. With the field advancing with new clinical trials every other day, the need for a reliable and reproducible potency test for these therapeutic immune cells has grown ever since. The industry “gold standard” to test effector function of immune cells is the Chromium release assay, which was developed in the 1960s and which is still in use, even with concerns due to the use of radioactive material and variability caused by target tumor cells. The alternative available is the Calcein-based assay, which still has a lot of variability caused by the use of different tumor targets and due to the entrapment of Calcein in apoptotic bodies of tumor targets.

There have been other efforts to develop different ways to see effector function of these immune cells visually, but these methods still use target tumor cells. Other surrogate methods to check the effector function of immune cells is to check the cytokine produced by these cells, for which all conventional methods use target tumor cells to induce cytokine production from immune cells. Use of the target tumor cells adds biological variability to all of these tests due to variability between tumor cell types. Also, these assays require a tedious setup, which introduces batch effect in these assays. Batch effect is caused by target cell conditions, person to person variability in loading of the plate, plate conditions, variability in various reagents, readout variability, etc. There is a clear need for an immune cell potency assay that can remove all of these variabilities and produce reliable and reproducible results.

A reliable and reproducible potency assay is required to evaluate the quality of immune cell therapy products. The approval process is intensely regulated and the drug developers will be required to submit a substantial amount of information regarding the drug product to the regulatory authorities in order to obtain approval. This may include information regarding the potency of the drug product and assays to determine this potency. As required by the FDA (21 CFR 610.10), potency of the cell therapy product should be indicated by appropriate tests to show effector function of these therapeutic immune cells to show potency, which would be measuring relevant cytokine production by these immune cells.

### SUMMARY

The details of one or more embodiments of the invention are set forth in the accompanying drawings and the description below. Other features, objects, and advantages of the invention will be apparent from the description and drawings, and from the claims.

In one aspect, disclosed herein are methods of assaying the potency of an immune cell (such as, for example, a T-cell, a macrophage, a NK cell, NK T cell, CAR T cell, and/or CAR NK cell), comprising contacting an immune cell with an effective amount of a plasma membrane particle, a liposome (including artificial liposomes), or an exosome (including, but not limited to engineered exosomes) and detecting the amount of one or more cytokines (such as, for example, IL-2, IL-6, IFN-γ, TNF-α, BAFF/TNFSF13B, CD163, CD30/TNFRSF8, Chitinase 3-like 1, gp130, IFN-α2, IL-6Rα, IL-8, IL-10, IL-11, IL-12(p40), IL-12(p70), IL-20, IL-22, IL-26, IL-29/IFN-11, IL-32, IL-34, IL-35, MMP-1, Osteocalcin, OPN, Pentraxin-3, TNF-R1, TNF-R2, TSLP, GM-CSF, MIP-160 , MIP-1β, RANTES, and/or TWEAK/TNFSF12) produced by the immune cell. In one aspect, the method can further comprise comparing the amount of cytokine produced to the cytokine potency level required for use of the immune cell in immunotherapy.

Also disclosed herein are methods of assaying the potency of an immune cell of any preceding aspect, wherein the amount of cytokine is detected using an immunoassay (such as, for example, ELISA, intracellular cytokine staining, ELISpot, flow cytometry, Luminex xMAP®, quantitative PCR (including, but not limited to qRT-PCR), and/or bead array).

In one aspect, disclosed herein are methods of assaying the potency of an immune cell of any preceding aspect wherein the immune cell is contacted with an effective amount of a plasma membrane particle, a liposome, or an exosome (including, but not limited to engineered exosomes) for at least 5, 6, 7, 8, 9, 10, 15, 20, 25, 30, 35, 40, 45, 50, 55, 60, 65, 70, 75, 80, 85, 90, 95, 100, 105, 110, 115, 120, 150 minutes, 3, 4, 5,6 7, 8, 9, 10, 11, 12, 13, 14, 15, 16, 17, 18, 19, 20, 21, 22, 23, 24, 30, 32, 36, 42, 48, 60 hours, 3, 4, 5, 6, 7, 8, 9, 10, 11, 12, 13, 14, 15, 16, 17, 18, 19, 20, 21, 22, 23, 24, 25, 26, 27, 28, 29, 30, 31, 45, 60, 61, 62 days, 3, 4, 5, or 6 months.

Also disclosed herein are methods of assaying the potency of an immune cell of any preceding aspect, wherein the plasma membrane particle, the liposome, or the exosome (including, but not limited to engineered exosomes) is provided at a concentration of 5 μg/mL to 1000 μg/mL, including, but not limited to a concentration of 50 μg/mL to 400 μg/mL.

In one aspect, disclosed herein are kits for assaying the potency of an immune cell (such as, for example, a T-cell, a macrophage, a NK cell, NK T cell, CAR T cell, and/or CAR NK cell), comprising a container (such as, for example, a microcentrifuge tube) including an effective amount of a plasma membrane particle and/or an exosome (including, but not limited to engineered exosomes) and a buffer suitable for immune cells. In some aspect, the kit can further comprise instructions for using the kit to stimulate cytokine production by an immune cell

Also disclosed herein are kits for assaying the potency of an immune cell of any preceding aspect, wherein the plasma membrane particle, the liposome, or the exosome (including, but not limited to engineered exosomes) is provided at a concentration of 5 μg/mL to 1000 μg/mL, including, but not limited to a concentration of 50 μg/mL to 400 μg/mL.

In one aspect, disclosed herein are immunotherapy method comprising a) performing the method of assaying the potency of an immune cell (such as, for example, a T-cell, a macrophage, a NK cell, NK T cell, CAR T cell, and/or CAR NK cell) of any preceding aspect on multiple immune cells to determine the potency of each immune cell; b) selecting at least one potent immune cell based on the amount of cytokine (such as, for example, IL-2, IL-6, IFN-γ, TNF-α, BAFF/TNFSF13B, CD163, CD30/TNFRSF8, Chitinase 3-like 1, gp130, IFN-α2, IL-6Rα, IL-8, IL-10, IL-11, IL-12(p40), IL-12(p70), IL-20, IL-22, IL-26, IL-29/IFN-11, IL-32, IL-34, IL-35, MMP-1, Osteocalcin, OPN, Pentraxin-3, TNF-R1, TNF-R2, TSLP, GM-CSF, MIP-1α, MIP-1β, RANTES, and/or TWEAK/TNFSF12) detected; and c) administering a therapeutically effective amount of the potent immune cell to a subject in need thereof as an immunotherapeutic. In one aspect, the method can further comprise extracting the multiple immune cells from an allogeneic or autologous donor prior to assaying the potency of the immune cell.

Also disclosed herein are immunotherapy methods of any preceding aspect further comprising expanding the at least one potent immune cell prior to delivering a therapeutically effective amount of the potent immune cell.

In one aspect, disclosed herein are immunotherapy methods of any preceding aspect further comprising directing the multiple immune cells or the potent immune cell to respond to a specified antigen.

Also disclosed herein are immunotherapy methods of any preceding aspect further comprising genetically altering the multiple immune cells or the potent immune cell to present a chimeric antigen receptor.

In one aspect, disclosed herein are methods of treating, inhibiting, reducing, preventing, and/or ameliorating a cancer and/or metastasis in a subject comprising a) obtaining one or more immune cells (such as, for example, a T-cell, a macrophage, a NK cell, NK T cell, CAR T cell, and/or CAR NK cell) obtained from an allogeneic or autologous donor); b) contacting an immune cell with an effective amount of a plasma membrane particle, a liposome, or an exosome (including, but not limited to engineered exosomes); c) detecting the amount of a cytokine (such as, for example, IL-2, IL-6, IFN-γ, TNF-α, BAFF/TNFSF13B, CD163, CD30/TNFRSF8, Chitinase 3-like 1, gp130, IFN-α2, IL-6Rα, IL-8, IL-10, IL-11, IL-12(p40), IL-12(p70), IL-20, IL-22, IL-26, IL-29/IFN-11, IL-32, IL-34, IL-35, MMP-1, Osteocalcin, OPN, Pentraxin-3, TNF-R1, TNF-R2, TSLP, GM-CSF, MIP-1α, MIP-1β, RANTES, and/or TWEAK/TNFSF12) produced by the immune cell; d) selecting at least one potent immune cell based on the amount of cytokine detected; and e) administering to the subject a therapeutically effective amount of the potent immune cell. In some aspect, the method can further comprise extracting the immune cell from an autologous or allogeneic donor.

Also disclosed herein are methods of treating, inhibiting, reducing, preventing, and/or ameliorating a cancer and/or metastasis of any preceding aspect further comprising expanding the at least one potent immune cell prior to delivering a therapeutically effective amount of the at least one potent immune cell.

Also disclosed herein are methods of determining the identity (such as, for example, differentiating Th1, Th2, Th3, Th9, Th17, effector memory T (Tem) cells, central memory T (Tcm) cells, γδT cells, or regulatory T (Treg) cells, resting NK cells, expanded NK cells) of at least one immune cell or a population of cells on the basis of the cytokines signature associated with that cell type.

### DESCRIPTION OF DRAWINGS

FIG. 1 provides a plot showing the correlation between NK cell cytokine release induced by exosomes (K562 cell-derived) versus NK cell cytokine release induced by PHA (in pg/million cells/hr).

FIG. 2 provides a plot showing the correlation between freshly isolated NK cell cytokine release (induced by K562 exosomes) versus expanded NK cell cytokine release induced by exosomes (in pg/million cells/hr).

FIG. 3 shows total cytokine concentration upon exposure to 4 different concentrations of exosome (60, 100, 200, and 400 mg/mL).

FIG. 4 shows the dose correlation of two different exosome concentrations.

### DETAILED DESCRIPTION

The present invention provides a method of determining the potency of an immune cell that includes contacting an immune cell with an effective amount of an exosome and detecting the amount of a cytokine produced by the immune cell. While the disclosure is given in the context of cancer immunotherapies, the concepts and innovations disclosed herein may be applied to immunotherapies for other diseases and disorders. For example, an immune cell used in immunotherapy against autoimmune disease, inflammatory diseases or disorders, viral diseases and/or bacterial infections can also be tested for potencies using the assays disclosed herein.

### Definitions

For clarification in understanding and ease in reference a list of terms used throughout the brief description section and the remainder of the application has been compiled here. Some of the terms are well known throughout the field and are defined here for clarity, while some of the terms are unique to this application and therefore have to be defined for proper understanding of the application.

As used in the specification and claims, the singular form “a,” “an,” and “the” include plural references unless the context clearly dictates otherwise. For example, the term “a cell” includes a plurality of cells, including mixtures thereof Where the plural form is used herein, it generally includes the singular.

Ranges can be expressed herein as from “about” one particular value, and/or to “about” another particular value. When such a range is expressed, another embodiment includes from the one particular value and/or to the other particular value. Similarly, when values are expressed as approximations, by use of the antecedent “about,” it will be understood that the particular value forms another embodiment. It will be further understood that the endpoints of each of the ranges are significant both in relation to the other endpoint, and independently of the other endpoint. Recitations of numerical ranges by endpoints include all numbers subsumed within that range (e.g., 1 to 5 includes 1, 1.5, 2, 2.75, 3, 3.80, 4, 5, etc.). It is also understood that there are a number of values disclosed herein, and that each value is also herein disclosed as “about” that particular value in addition to the value itself. For example, if the value “10” is disclosed, then “about 10” is also disclosed. It is also understood that when a value is disclosed that “less than or equal to” the value, “greater than or equal to the value” and possible ranges between values are also disclosed, as appropriately understood by the skilled artisan. For example, if the value “10” is disclosed the “less than or equal to 10” as well as “greater than or equal to 10” is also disclosed. It is also understood that throughout the application, data is provided in a number of different formats and that this data represents endpoints and starting points, and ranges for any combination of the data points. For example, if a particular data point “10” and a particular data point 15 are disclosed, it is understood that greater than, greater than or equal to, less than, less than or equal to, and equal to 10 and 15 are considered disclosed as well as between 10 and 15. It is also understood that each unit between two particular units are also disclosed. For example, if 10 and 15 are disclosed, then 11, 12, 13, and 14 are also disclosed.

As used herein, the term “comprising” is intended to mean that the compositions and methods include the recited elements, but not excluding others. “Consisting essentially of” when used to define compositions and methods, shall mean excluding other elements of any essential significance to the combination. Thus, a composition consisting essentially of the elements as defined herein would not exclude trace contaminants from the isolation and purification method and pharmaceutically acceptable carriers, such as phosphate buffered saline, preservatives, and the like. “Consisting of” shall mean excluding more than trace elements of other ingredients and substantial method steps for administering the compositions of this invention. Embodiments defined by each of these transition terms are within the scope of this invention.

An “increase” can refer to any change that results in a greater amount of a symptom, disease, composition, condition or activity. An increase can be any individual, median, or average increase in a condition, symptom, activity, composition in a statistically significant amount. Thus, the increase can be a 1, 2, 3, 4, 5, 6, 7, 8, 9, 10, 15, 20, 25, 30, 35, 40, 45, 50, 55, 60, 65, 70, 75, 80, 85, 90, 95, or 100% increase so long as the increase is statistically significant.

A “decrease” can refer to any change that results in a smaller amount of a symptom, disease, composition, condition, or activity. A substance is also understood to decrease the genetic output of a gene when the genetic output of the gene product with the substance is less relative to the output of the gene product without the substance. Also for example, a decrease can be a change in the symptoms of a disorder such that the symptoms are less than previously observed. A decrease can be any individual, median, or average decrease in a condition, symptom, activity, composition in a statistically significant amount. Thus, the decrease can be a 1, 2, 3, 4, 5, 6, 7, 8, 9, 10, 15, 20, 25, 30, 35, 40, 45, 50, 55, 60, 65, 70, 75, 80, 85, 90, 95, or 100% decrease so long as the decrease is statistically significant.

“Inhibit,” “inhibiting,” and “inhibition” mean to decrease an activity, response, condition, disease, or other biological parameter. This can include but is not limited to the complete ablation of the activity, response, condition, or disease. This may also include, for example, a 10% reduction in the activity, response, condition, or disease as compared to the native or control level. Thus, the reduction can be a 10, 20, 30, 40, 50, 60, 70, 80, 90, 100%, or any amount of reduction in between as compared to native or control levels.

By “reduce” or other forms of the word, such as “reducing” or “reduction,” is meant lowering of an event or characteristic (e.g., tumor growth). It is understood that this is typically in relation to some standard or expected value, in other words it is relative, but that it is not always necessary for the standard or relative value to be referred to. For example, “reduces tumor growth” means reducing the rate of growth of a tumor relative to a standard or a control.

By “prevent” or other forms of the word, such as “preventing” or “prevention,” is meant to stop a particular event or characteristic, to stabilize or delay the development or progression of a particular event or characteristic, or to minimize the chances that a particular event or characteristic will occur. Prevent does not require comparison to a control as it is typically more absolute than, for example, reduce. As used herein, something could be reduced but not prevented, but something that is reduced could also be prevented. Likewise, something could be prevented but not reduced, but something that is prevented could also be reduced. It is understood that where reduce or prevent are used, unless specifically indicated otherwise, the use of the other word is also expressly disclosed.

The term “therapeutically effective” is intended to qualify the number or amount of an active agent (such as immunotherapeutic cells) which will achieve the goal of decreasing disease severity while avoiding adverse side effects such as those typically associated with alternative therapies. A therapeutically effective amount may be administered in one or more doses. Treatments that are therapeutically effective include treatments that improve a subject's quality of life even if they do not improve the disease outcome per se

An “effective amount” generally means an amount which provides the desired local or systemic effect, e.g., effective to stimulate cytokine formation, including achieving the specific desired effects described in this application. For example, an effective amount is an amount sufficient to effectuate a beneficial or desired clinical result.

The term “subject” refers to any individual who is the target of administration or treatment. The subject can be a vertebrate, for example, a mammal. In one aspect, the subject can be human, non-human primate, bovine, equine, porcine, canine, or feline. The subject can also be a guinea pig, rat, hamster, rabbit, mouse, or mole. Thus, the subject can be a human or veterinary patient. The term “patient” refers to a subject under the treatment of a clinician, e.g., physician.

The term “therapeutically acceptable carrier” means a carrier or excipient that is useful in preparing a composition that is generally safe and non-toxic, and includes a carrier that is acceptable for veterinary and/or human use. Intravenous delivery methods will utilize a therapeutically acceptable carrier that is physiologically balanced (for example, at an osmotic and pH level that is safe for intravenous use). As used herein, the term “therapeutically acceptable carrier” encompasses any of the standard carriers, such as saline, Ringers, a phosphate buffered saline solution, water, dextrose in water, and emulsions, such as an oil/water or water/oil emulsion, and various types of wetting agents. As used herein, the term “carrier” encompasses any excipient, diluent, filler, salt, buffer, stabilizer, solubilizer, lipid, stabilizer, or other material well known in the art for use in therapeutic formulations. The therapeutically acceptable carrier also can include preservatives (including cryopreservatives), such as those that would preserve the viability and/or potency of an immune cell. A “therapeutically acceptable carrier” as used in the specification and claims includes both one and more than one such carrier.

The term “treatment” refers to the medical management of a patient with the intent to cure, ameliorate, stabilize, or prevent a disease, pathological condition, or disorder. This term includes active treatment, that is, treatment directed specifically toward the improvement of a disease, pathological condition, or disorder, and also includes causal treatment, that is, treatment directed toward removal of the cause of the associated disease, pathological condition, or disorder. In addition, this term includes palliative treatment, that is, treatment designed for the relief of symptoms rather than the curing of the disease, pathological condition, or disorder; preventative treatment, that is, treatment directed to minimizing or partially or completely inhibiting the development of the associated disease, pathological condition, or disorder; and supportive treatment, that is, treatment employed to supplement another specific therapy directed toward the improvement of the associated disease, pathological condition, or disorder.

“Administration” to a subject includes any route of introducing or delivering to a subject an agent. Administration can be carried out by any suitable route, including oral, topical, intravenous, subcutaneous, transcutaneous, transdermal, intramuscular, intra joint, parenteral, intra-arteriole, intradermal, intraventricular, intracranial, intraperitoneal, intralesional, intranasal, rectal, vaginal, by inhalation, via an implanted reservoir, parenteral (e.g., subcutaneous, intravenous, intramuscular, intra-articular, intra-synovial, intrasternal, intrathecal, intraperitoneal, intrahepatic, intralesional, and intracranial injections or infusion techniques), and the like. “Concurrent administration”, “administration in combination”, “simultaneous administration” or “administered simultaneously” as used herein, means that the compounds are administered at the same point in time or essentially immediately following one another. In the latter case, the two compounds are administered at times sufficiently close that the results observed are indistinguishable from those achieved when the compounds are administered at the same point in time. “Systemic administration” refers to the introducing or delivering to a subject an agent via a route which introduces or delivers the agent to extensive areas of the subject's body (e.g. greater than 50% of the body), for example through entrance into the circulatory or lymph systems. By contrast, “local administration” refers to the introducing or delivery to a subject an agent via a route which introduces or delivers the agent to the area or area immediately adjacent to the point of administration and does not introduce the agent systemically in a therapeutically significant amount. For example, locally administered agents are easily detectable in the local vicinity of the point of administration, but are undetectable or detectable at negligible amounts in distal parts of the subject's body. Administration includes self-administration and the administration by another.

“Treat,” “treating,” “treatment,” and grammatical variations thereof as used herein, include the administration of a composition with the intent or purpose of partially or completely preventing, delaying, curing, healing, alleviating, relieving, altering, remedying, ameliorating, improving, stabilizing, mitigating, and/or reducing the intensity or frequency of one or more a diseases or conditions, a symptom of a disease or condition, or an underlying cause of a disease or condition. Treatments according to the invention may be applied preventively, prophylactically, pallatively or remedially. Prophylactic treatments are administered to a subject prior to onset (e.g., before obvious signs of cancer), during early onset (e.g., upon initial signs and symptoms of cancer), or after an established development of cancer. Prophylactic administration can occur for day(s) to years prior to the manifestation of symptoms of a disease or an infection.

Throughout this application, various publications are referenced. The disclosures of these publications in their entireties are hereby incorporated by reference into this application in order to more fully describe the state of the art to which this pertains. The references disclosed are also individually and specifically incorporated by reference herein for the material contained in them that is discussed in the sentence in which the reference is relied upon.

### Immune Potency Assay

In one aspect, the invention provides a method of determining the potency of an immune cell. The method includes the steps of contacting an immune cell with an effective amount of a plasma membrane particle, a liposome, or an exosome (for example, a cancer cell exosome or engineered exosome), and detecting the amount of a cytokine produced by the immune cell. For example, the immune cell can be contacted with a plasma membrane particle or exosome (including, but not limited to engineered exosomes) by suspending the exosome in a cell medium and exposing the immune cells to the cell medium.

In some embodiments, the method includes the step of comparing the amount of cytokine produced to the cytokine potency level required for use of the immune cell in immunotherapy. A potency assay serves to characterize the product (i.e., immune cells), to monitor lot-to-lot consistency and to assure stability of the product, and should therefore be sufficiently sensitive to detect differences which may impact mechanism of action and function of the product and are thereby of potential clinical importance. The assay can also be used as a predictive biomarker or pharmacodynamic assay for cell-mediated immunotherapy. It is preferable for the potency assay bears the closest possible relationship to the putative physiological/pharmacological activity of the product. The potency assay described herein provides the ability to measure potency value within the product specifications; high sensitivity for detection of differences of potential clinical importance; close relationship with the mechanism of action and putative physiological/pharmacological activity of the product. Preferably, the potency assay also satisfies the following secondary criteria: sufficiently low intra- and inter-assay variation (to obtain precision needed to support product specifications); sufficient robustness; and amenable to high-throughput analysis. In some embodiments, the assay is used as a clinical assay to quantify T cell, macrophage, NK cell, NK T cell, CAR T cell, and/or CAR NK cell function (diagnostic for NK cell immune deficiency, biomarker for monitoring immunosuppressant or immune-activator effectiveness).

As noted above, the disclosed methods provide for determining the potency of an immune cell. Immune cells, as defined herein, are any cells of the immune system that produce cytokines (i.e., cytokine- producing immune cells). Examples of cytokine-producing immune cells include lymphocytes, neutrophils, macrophages, and natural killer cells. Lymphocytes include both B-cells and T-cells (including CD4 and CD8 T cells). In one aspect, the immune cell can comprise a tumor infiltrating lymphocyte (TIL), T cell, natural killer (NK) cell, NK T cell, chimeric antigen receptor (CAR) T cell, and/or CAR NK. The immune cells can be obtained from cell culture, or can be obtained from a subject (such as, for example, an allogenic donor or autologous donor).

In some embodiments, the immune cell is a T-cell. T-cells play a central role in cell-mediated immunity, and can be distinguished from other lymphocytes, such as B cells and natural killer cells, by the presence of a T-cell receptor on the cell surface. Examples of T-cells include T helper cells (TH cells), cytotoxic T cells (TC cells), memory T cells, regulatory or “suppressor” T cells, and Natural killer T cells (NKT cells, which are distinct from NK cells and recognize a glycolipid antigen rather than peptides presented by the MHC molecule. Different types of T-cells differ from each other in their pattern of cytokine production). T cells can be CD4 or CD8 T cells. Additionally, T cells can comprise chimeric antigen receptor (CAR) T cells or tumor infiltrating lymphocytes (TILs).

In some embodiments, the immune cell is an NK cell. Natural Killer Cells are a type of cytotoxic lymphocyte of the immune system. NK cells provide rapid responses to virally infected cells and respond to transformed cells. Typically, immune cells detect peptides from pathogens presented by Major Histocompatibility Complex (MHC) molecules on the surface of infected cells, triggering cytokine release, causing lysis or apoptosis. NK cells are unique, however, as they have the ability to recognize stressed cells regardless of whether peptides from pathogens are present on MHC molecules. They were named “natural killers” because of the initial notion that they do not require prior activation in order to kill target. NK cells are large granular lymphocytes (LGL) and are known to differentiate and mature in the bone marrow from where they then enter into the circulation. In some aspect, the NK cell can be a CAR NK cell.

Thus, in one aspect, disclosed herein are methods of assaying the potency of an immune cell (such as, for example, a T-cell, a macrophage, a NK cell, NK T cell, CAR T cell, and/or CAR NK cell), comprising contacting an immune cell with an effective amount of a plasma membrane particle, a liposome, or an exosome (including, but not limited to engineered exosomes) and detecting the amount of one or more cytokines produced by the immune cell. In one aspect, the method can further comprise comparing the amount of cytokine produced to the cytokine potency level required for use of the immune cell in immunotherapy.

The assay includes the step of detecting the amount of a cytokine produced by the immune cell after stimulating the immune cells with exosome. As used herein, the term “cytokine” refers to a small protein (˜5-20 kDa) that is important in cell signaling, and in particular immunomodulation that can be produced by an immune cell. Examples of cytokines include chemokines, interferons, interleukins, lymphokines, and tumor necrosis factors. The cytokines detected can include cytokines known to be produced by the immune cells being evaluated, or the detection can encompass a wider variety of cytokines, including cytokines not known to be produced by the immune cells.

In some embodiments, the cytokines being detected include cytokines known to be produced by T-cells or Natural Killer cells. In some embodiments, the cytokines include those known to be produced by T-cells. T-cells include Th1 and Th2 cells; Th1 cells predominantly produce interferon (IFN)-γ (IFN-γ), tumor necrosis factor (TNF)-α (TNF-α), and IL-2; Th2 cells produce interleukin (IL)-2 (IL-2), IL-4, IL-5, IL-6, IL-9, IL-13, and IL-22. Examples of cytokines produced by stimulated Natural Killer cells include IL-1α, IL-1β, IL- 2, IL-5, IL-8, IL-10, IL-13, IFN-γ, TNF-α, granulocyte-macrophage colony-stimulating factor (GM-CSF), leukemia inhibitory factor (LIF), and the chemokines macrophage inflammatory protein (MIP)-1α (MIP-1α), MIP-1β, and RANTES. Other cytokines useful to determine the potency of an immune cell include, but are not limited to B cell activating factor/ tumor necrosis factor (TNF) ligand superfamily member 13B (BAFF/TNFSF13B), cluster of differentiation (CD) 163 (CD163), CD30/TNFRSF8, Chitinase 3-like 1, gp130, IFN-α2, IL-6Rα, IL-11, IL-12(p40), IL-12(p70), IL-20, IL-26, IL-29/IFN-11, IL-32, IL-34, IL-35, matrix metalloproteinase-1 (MMP-1), Osteocalcin, Osteopontin (OPN), Pentraxin-3, tumor necrosis factor (TNF)-receptor 1 (TNF-R1), TNF-R2, thymic stromal lymphopoetin (TSLP), or TNF-related weak inducer of apoptosis (TWEAK)/TNF superfamily member 12 (TWEAK/TNFSF12). Thus, in one aspect, disclosed herein are methods of assaying the potency of an immune cell (such as, for example, a T-cell, a macrophage, a NK cell, NK T cell, CAR T cell, and/or CAR NK cell), comprising contacting an immune cell with an effective amount of a plasma membrane particle, a liposome, or an exosome (including, but not limited to engineered exosomes) and detecting the amount of one or more cytokines (such as, for example, IL-2, IL-6, IFN-γ, TNF-α, BAFF/TNFSF13B, CD163, CD30/TNFRSF8, Chitinase 3-like 1, gp130, IFN-α2, IL-6Rα, IL-8, IL-10, IL-11, IL-12(p40), IL-12(p70), IL-20, IL-22, IL-26, IL-29/IFN-11, IL-32, IL-34, IL-35, MMP-1, Osteocalcin, OPN, Pentraxin-3, TNF-R1, TNF-R2, TSLP, GM-CSF, LIF, MIP-1α, MIP-1β, RANTES and/or TWEAK/TNFSF12) produced by the immune cell. disclosed herein are methods of assaying the potency of an immune cell (such as, for example, a T-cell, a macrophage, a NK cell, NK T cell, CAR T cell, and/or CAR NK cell), comprising contacting an immune cell with an effective amount of a plasma membrane particle, a liposome, or an exosome (including, but not limited to engineered exosomes) and detecting the amount of one or more cytokines (such as, for example, IL-2, IL-6, IFN-γ, TNF-α, BAFF/TNFSF13B, CD163, CD30/TNFRSF8, Chitinase 3-like 1, gp130, IFN-α2, IL-6Rα, IL-8, IL-10, IL-11, IL-12(p40), IL-12(p70), IL-20, IL-22, IL-26, IL-29/IFN-11, IL-32, IL-34, IL-35, MMP-1, Osteocalcin, OPN, Pentraxin-3, TNF-R1, TNF-R2, TSLP, GM-CSF, MIP-1α, MIP-1β, RANTES, and/or TWEAK/TNFSF12) produced by the immune cell. In one aspect, the method can further comprise comparing the amount of cytokine produced to the cytokine potency level required for use of the immune cell in immunotherapy. In some embodiments, the levels of a plurality of cytokines are determined. In further embodiments, the cytokine is selected from the group consisting of interleukin-2, interleukin-6, and interferon-y.

The assay includes the step of detecting the amount of a cytokine produced by the immune cell. A wide variety of methods are known to those skilled in the art for detecting cytokines, which can vary depending on the cytokine being detected. In some embodiments, a method or methods can be used to detect and/or quantify the presence of a plurality of different cytokines. Cytokines can be detected by, for example, the use of specific reagent kits or immunoassays. Cytokines can be detected using kits available from commercial providers such as Miltenyi Biotec™, Luminex, and Thermo Fisher scientific™. Examples of kits suitable for detecting cytokines are the rapid cytokine inspector (CD4/CD8) kit, or the MACSPlex cytokine T/NK kit, which can detect cytokines formed by either T-cells or NK cells, both of which are sold by Miltenyi Biotec™.

In some embodiments, the amount of cytokine is detected using an immunoassay. Immunoassays come in many different formats and variations. Immunoassays may be run in multiple steps with reagents being added and washed away or separated at different points in the assay. Immunoassays include heterogeneous immunoassays, which include multiple steps, and homogenous immunoassays, which involve simply mixing the reagents and sample and making a physical measurement. Immunoassays often make use of a calibrator, which is a solution known to contain the analyte in question, and the concentration of that analyte is generally known. Comparison of an assay's response to a real sample against the assay's response produced by the calibrators makes it possible to interpret the signal strength in terms of the presence or concentration of analyte in the sample. Types of immunoassays include competitive, homogenous immunoassays, competitive heterogenous immunoassays, one-site non-competitive immunoassays, and two-site noncompetitive immunoassays. Immunoassays also include Enzyme-linked immunosorbent assays (ELISA), lateral flow immunoassays, enzyme-linked immunosorbent spot (ELlspot) assays, flow cytometry, intracellular cytokine staining, antibody array assays and bead-based assays, magnetic immunoassays, radioimmunoassays, and quantitative PCR (including, but not limited to qRT-PCR). In one aspect, the assay comprises a Luminex xMAP®.

The method of determining the potency of an immune cell includes the step of contacting an immune cell with an effective amount of a plasma membrane particle and/or an exosome (such as for example, an engineered exosome). Plasma membrane (PM) particles are vesicles made from the plasma membrane of a cell or artificially made (i.e., liposomes). A PM particle can contain a lipid bilayer or simply a single layer of lipids. A PM particle can be prepared in single lamellar, multi-lamellar, or inverted form. PM particles can be prepared from Fc-bound feeder cells as described herein, using known plasma membrane preparation protocols or protocols for preparing liposomes such as those described in U.S. Pat. No. 9,623,082, the entire disclosure of which is herein incorporated by reference. In certain aspects, PM particles as disclosed herein range in average diameter from about 170 to about 300 nm.

Exosomes are cell-derived vesicles that are present in many and perhaps all eukaryotic fluids. Exosomes contain RNA, proteins, lipids and metabolites that is reflective of the cell type of origin. The reported diameter of exosomes is between 30 and 100 nm. Exosomes are either released from the cell when multivesicular bodies fuse with the plasma membrane or released directly from the plasma membrane. In some embodiments, exosomes are obtained from cancer cells. In some embodiments, the exosomes are leukemic cell exosomes. While this disclosure is given in the context of using exosomes to determine the potency of an immune cell, other extracellular vesicles may also be used to determine the potency of an immune cell. As used herein, the term “extracellular vesicle” includes, but is not limited to, all vesicles released from cells by any mechanism. “Extracellular vesicles” includes exosomes which are released from multivesicular bodies and microvesicles that are shed from the cell surface. “Extracellular vesicles” includes vesicles created by exocytosis or ectocytosis. “Extracellular vesicles” encompasses exosomes released from multivesicular bodies, vesicles released by reverse budding, fission of membrane(s), multivesicular endosomes, ectosomes, microvesicles, microparticles, and vesicles released by apoptotic bodies, and hybrid vesicles containing plasma membrane components. Extracellular vesicles can contain proteins, nucleic acids, lipids, and other molecules common to the originating cell.

In one aspect, the plasma membrane particles, or exosomes can be purified from feeder cells that stimulate immune cells (such as, for example NK cells). Immune cell stimulating feeder cells for use in the claimed invention, for use in making the plasma membrane particles or making the exosomes disclosed herein can be either irradiated autologous or allogeneic peripheral blood mononuclear cells (PBMCs) or nonirradiated autologous or allogeneic PBMCs, RPMI8866, HFWT, 721.221, K562 cells, EBV-LCLs, T cells transfected with one or more membrane bound IL-21, membrane bound IL-15, membrane bound 4-1BBL, membrane bound OX40L and/or membrane TNF-α, (such as for example, T cells transfected with membrane bound IL-21, T cells transfected with membrane bound 4-1BBL, T cells transfected with membrane bound IL-15 and 4-1BBL, T cells transfected with membrane bound IL-21 and 4-1BBL), NK cells (including, but not limited to PBMCs, RPMI8866, NK-92, NK-92MI, NK-YTS, NK, NKL, ML, ML C.2, NK 3.3, NK-YS, HFWT, K562 cells) transfected with membrane bound IL-21, NK cells (including, but not limited to PBMCs, RPMI8866, NK-92, NK-92MI, NK-YTS, NK, NKL, ML, ML C.2, NK 3.3, NK-YS, HFWT, K562 cells) transfected with membrane bound 4-1BBL, NK cells (including, but not limited to PBMCs, RPMI8866, NK-92, NK-92MI, NK-YTS, NK, NKL, ML, ML C.2, NK 3.3, NK-YS, HFWT, K562 cells) transfected with membrane bound IL-15 and 4-1BBL , or NK cells (including, but not limited to PBMCs, RPMI8866, NK-92, NK-92MI, NK-YTS, NK, NKL, ML, ML C.2, NK 3.3, NK-YS, HFWT, K562 cells) transfected with membrane bound IL-21 and 4-1BBL as well as other non-HLA or low-HLA expressing cell lines or patient derived primary tumors.

The plasma membrane particles and/or exosomes used in the disclosed methods can further comprise additional effector agents to expand and/or activate immune cells (such as, for example, NK cells). Thus, in one aspect disclosed herein are methods of assaying the potency of an immune cells, wherein the feeder cells used to generate the disclosed exosomes or plasma membrane particles further comprise at least one additional immune cell effector agent on its cell surface, wherein the at least one additional immune cell effector agent is a cytokine, an adhesion molecule, or an immune cell activating agent (such as, for example, 4-1BBL, IL-2, IL-12, IL-15, IL-18, IL-21, MICA, LFA-1, 2B4, CCR7, OX40L, UBLP2, BCM1/SLAMF2, NKG2D agonists, CD155, CD112, Jagged', Jagged2, Delta-1, Pref-1, DNER, Jedi, SOM-11, wingless, CCN3, MAGP2, MAGP1, TSP2, YB-1, EGFL7, CCR7, DAP12, and DAP10, Notch ligands, NKp46 agonists, NKp44 agonists, NKp30 agonists, other NCR agonists, CD16 agonists). In one aspect the at least one additional immune cell effector agent comprises IL-21, 4-1BBL, IL-15, IL-21 and 4-1BBL, IL-21 and IL-15, or IL-15 and 4-1BBL. Accordingly, in one aspect, the plasma membrane particles and exosomes generated by said feeder cells and used in the methods of assaying the potency of immune cells disclosed herein can comprise membrane bound versions of any combination of the immune cell activating agents (such as, for example, 4-1BBL, IL-2, IL-12, IL-15, IL-18, IL-21, MICA, LFA-1, 2B4, CCR7, OX40L, UBLP2, BCM1/SLAMF2, NKG2D agonists, CD155, CD112, Jagged1, Jagged2, Delta-1, Pref-1, DNER, Jedi, SOM-11, wingless, CCN3, MAGP2, MAGP1, TSP2, YB-1, EGFL7, CCR7, DAP12, and DAP10, Notch ligands, NKp46 agonists, NKp44 agonists, NKp30 agonists, other NCR agonists, CD16 agonists). For example, the exosomes or plasma membrane particles can have IL-15, IL-21, and/or 4-1BBL on their membrane.

It is understood and herein contemplated that the immune cells must be exposed to the particle or exosome for a period of time to be induced to produce cytokines. In one aspect, disclosed herein are methods of assaying the potency of an immune cell wherein the immune cell is contacted with an effective amount of a plasma membrane particle, a liposome, or an exosome (including, but not limited to engineered exosomes) for at least 5, 6, 7, 8, 9, 10, 15, 20, 25, 30, 35, 40, 45, 50, 55, 60, 65, 70, 75, 80, 85, 90, 95, 100, 105, 110, 115, 120, 150 minutes, 3, 4, 5,6 7, 8, 9, 10, 11, 12, 13, 14, 15, 16, 17, 18, 19, 20, 21, 22, 23, 24, 30, 32, 36, 42, 48, 60 hours, 3, 4, 5, 6, 7, 8, 9, 10, 11, 12, 13, 14, 15, 16, 17, 18, 19, 20, 21, 22, 23, 24, 25, 26, 27, 28, 29, 30, 31, 45, 60, 61, 62 days, 3, 4, 5, or 6 months.

Also disclosed herein are methods of assaying the potency of an immune cell of any preceding aspect, wherein the plasma membrane particle, the liposome, or the exosome (including, but not limited to engineered exosomes) is provided at a concentration of 5 μg/mL to 1000 μg/mL, In one aspect, the concentration of the particle or exosome is 5, 10, 15, 20, 25, 30, 35, 40, 45, 50, 55, 60, 65, 70, 75, 80, 85, 90, 95, 100, 110, 120, 125, 130, 140, 150, 160, 170, 175, 180, 190, 200, 225, 250, 275, 300, 325, 350, 375, 400, 425, 450, 475, 500, 550, 600, 650, 700, 750, 800, 850, 900, 950, or 1000 1000 μg/mL. In one aspect, the concentration of the exosome or particle is from about 50 μg/mL to 100 μg/mL, 50 μg/mL to 200 μg/mL, 50 μg/mL to 300 μg/mL, 50 μg/mL to 500 μg/mL, or 100 μg/mL to 500 μg/mL. Preferably the concentration of the exosome or particle is from about 50 μg/mL to 400 μg/mL.

In some embodiments, the immune cells are stimulated using exosomes from non-modified cancer cells, such as non-modified K562. However, in other embodiments, antigen-specific cells are stimulated using exosomes from antigen-expressing cells. For example, antigen- specific therapeutic cells (e.g., CAR-T cells, CAR-NK cells) can be stimulated with exosomes from antigen-expressing K562, or targeted cell engagers (Bi-specific engagers, BiTEs, BiKEs, TriNKETs) using antigen-expressing exosomes and patient blood cells.

In one aspect, it is understood and herein contemplated that the same cytokines produced to determine potency of an immune cell can also be used to identify the cells producing the cytokines. Immune cells have distinct expression profiles that well known in the art. Also disclosed herein are methods of determining the identity of at least one immune cell or a population of cells (such as, for example, differentiating Th1, Th2, Th3, Th9, Th17, effector memory T (Tem) cells, central memory T (Tcm) cells, γδT cells, or regulatory T (Treg) cells, resting NK cells, expanded NK cells) on the basis of the cytokines signature associated with that cell type. Accordingly, disclosed herein are methods of identifying an immune cell (such as, for example, a T-cell, a macrophage, a NK cell, NK T cell, CAR T cell, and/or CAR NK cell), comprising contacting an immune cell with an effective amount of a plasma membrane particle, a liposome, or an exosome (including, but not limited to engineered exosomes) and detecting the amount of one or more cytokines (such as, for example, IL-2, IL-6, IFN-γ, TNF-α, BAFF/TNFSF13B, CD163, CD30/TNFRSF8, Chitinase 3-like 1, gp130, IFN-α2, IL-6Rα, IL-8, IL-10, IL-11, IL-12(p40), IL-12(p70), IL-20, IL-22, IL-26, IL-29/IFN-11, IL-32, IL-34, IL-35, MMP-1, Osteocalcin, OPN, Pentraxin-3, TNF-R1, TNF-R2, TSLP, GM-CSF, MIP-1α, MIP-1β, RANTES, and/or TWEAK/TNFSF12) produced by the immune cell; wherein the identity of the immune cell is revealed based on the profile of cytokines expressed.

Kits for Evaluating Immune Cell Potency

Another aspect of the invention provides a kit for determining the potency of an immune cell (such as, for example, a T-cell, a macrophage, a NK cell, NK T cell, CAR T cell, and/or CAR NK cell), comprising a container including an effective amount of a particle or exosome (such as, for example, an exosome (including, but not limited to engineered exosomes) or plasma membrane particle) and a buffer suitable for immune cells. In some embodiments, the exosome in the kit is provided at a concentration of 5 μg/mL to 1000 μg/mL, In one aspect, the concentration of the particle or exosome is 5, 10, 15, 20, 25, 30, 35, 40, 45, 50, 55, 60, 65, 70, 75, 80, 85, 90, 95, 100, 110, 120, 125, 130, 140, 150, 160, 170, 175, 180, 190, 200, 225, 250, 275, 300, 325, 350, 375, 400, 425, 450, 475, 500, 550, 600, 650, 700, 750, 800, 850, 900, 950, or 1000 1000 μg/mL. In one aspect, the concentration of the exosome or particle is from about 50 μg/mL to 100 μg/mL, 50 μg/mL to 200 μg/mL, 50 μg/mL to 300 μg/mL, 50 μg/mL to 500 μg/mL, or 100 μg/mL to 500 μg/mL. Preferably the concentration of the exosome or particle is from about 50 μg/mL to 400 μg/mL. In some embodiments, the container is a microcentrifuge tube (such as, for example an Eppendorf microcentrifuge tube). Kits can also include a tool for obtaining a sample from a subject, such as a syringe to obtain a sample including one or more immune cells. A suitable buffer is RPMI.

The kits may also include the components required for conducting an immunoassay, such as a solid phase, to which the antibodies functioning as capture antibodies and/or detection antibodies in a sandwich immunoassay format are bound. The solid phase may be a material such as a magnetic particle, a bead, a test tube, a microtiter plate, a cuvette, a membrane, a scaffolding molecule, a quartz crystal, a film, a filter paper, a disc or a chip. The kit may also include a detectable label that can be or is conjugated to an antibody, such as an antibody functioning as a detection antibody. The detectable label can for example be a direct label, which may be an enzyme, oligonucleotide, nanoparticle chemiluminophore, fluorophore, fluorescence quencher, chemiluminescence quencher, or biotin. Test kits may optionally include any additional reagents needed for detecting the label.

The kit can further include instructions for using the kit to stimulate cytokine production by an immune cell in order to evaluate the potency of the immune cell. In some embodiments, the kit further includes instructions for using the amount of cytokine to determine the potency of the cell. Instructions included in kits can be affixed to packaging material or can be included as a package insert. While the instructions are typically written or printed materials, they are not limited to such. Any medium capable of storing such instructions and communicating them to an end user is contemplated by this disclosure. Such media include, but are not limited to, electronic storage media (e.g., magnetic discs, tapes, cartridges, chips), optical media (e.g., CD ROM), and the like. As used herein, the term “instructions” can include the address of an interne site that provides the instructions.

### Immunotherapy Methods

The method of determining the potency of an immune cell can be performed prior to the use of the immune cell as an immunotherapeutic agent. For example, the method of determining the potency of one or multiple immune cells can be performed as described above, after which at least one potent immune cell can be selected (based on the amount of cytokine detected) and a therapeutically effective amount of the potent immune cell can be delivered to a subject as an immunotherapeutic. Thus, In one aspect, disclosed herein are immunotherapy methods comprising a) performing the method of assaying the potency of an immune cell (such as, for example, a T-cell, a macrophage, a NK cell, NK T cell, CAR T cell, and/or CAR NK cell) as disclosed herein on multiple immune cells to determine the potency of each immune cell; b) selecting at least one potent immune cell based on the amount of cytokine (such as, for example, IL-2, IL-6, IFN-γ, TNF-α, BAFF/TNFSF13B, CD163, CD30/TNFRSF8, Chitinase 3-like 1, gp130, IFN-α2, IL-6Rα, IL-8, IL-10, IL-11, IL-12(p40), IL-12(p70), IL-20, IL-22, IL-26, IL-29/IFN-11, IL-32, IL-34, IL-35, MMP-1, Osteocalcin, OPN, Pentraxin-3, TNF-R1, TNF-R2, TSLP, GM-CSF, MIP-1α, MIP-1β, RANTES, and/or TWEAK/TNFSF12) detected; and c) administering a therapeutically effective amount of the potent immune cell to a subject in need thereof as an immunotherapeutic. In one aspect, the method can further comprise extracting the multiple immune cells from an allogeneic or autologous donor prior to assaying the potency of the immune cell.

In some embodiments, the immune cells are immunotherapeutic immune cells. Immunotherapeutic immune cells are those that are useful for treatment of diseases such as cancer. Becker et al., Cancer Immunol. Immunother 65, 477-484 (2016). The use of expanded NK cells for treatment of cancer has been described. Rezvani et al., Front Immunol., 6, 578 (2015). Because it is helpful to be able to administer large numbers of immune cells during immunotherapy, in some embodiments the immune cells are expanded immune cells. Expanded immune cells are those that are grown ex-vivo in order to grow a large number of immune cells. In some embodiments, the expanded immune cells are autologous cells that can be easily administered to a subject without provoking an immune response. However, in some embodiments, the expanded immune cells are allogeneic immune cells, in which their inherent alloreactivity can be a benefit. In further embodiments, the expanded immune cells are genetically engineered to include chimeric antigen receptors to help the immune cells target diseased tissue. Preparation of expanded immune cells includes activating and expanding the immune cells. Koepsell et al., Transfusion, 53(2):404-10 (2013). A number of cytokines (IL-2, IL-12, IL-15, IL-18, IL-21, type I IFNs, and TGF-β) have been shown to be useful for activating and expanding immune cells ex vivo. For example, in some embodiments, the NK cells being evaluated are IL-21 expanded NK cells. Accordingly, in one aspect, disclosed herein are immunotherapy methods further comprising expanding the at least one potent immune cell prior to delivering a therapeutically effective amount of the potent immune cell.

Expansion refers to the ex vivo proliferation of NK cells so that the population of NK cells is increased. NK cells can be expanded, for example, from peripheral blood mononuclear cells. However, NK cells can also be expanded from other types of cells, such as hematopoietic stem cells or progenitor cells. The initial blood or stem cells can be isolated from a variety of different sources, such placenta, umbilical cord blood, placental blood, peripheral blood, spleen or liver. Expansion occurs in a cell culture medium. Suitable cell culture mediums are known to those skilled in the art. The expanded cells can be a provided as a cell line, which is a plurality of cells that can be maintained in cell culture. Thus, in one aspect, disclosed herein are immunotherapy methods further comprising expanding the at least one potent immune cell prior to delivering a therapeutically effective amount of the potent immune cell. In some aspects, the immune cell has been extracted from a subject using known methods prior to performing the method of determining the potency of the immune cell. Alternatively, the immune cell can be sourced from expansion of a cell culture.

In some aspects, an immune cell is directed to respond to a specified antigen. The immune cell can be directed to respond prior to the method of determining its potency, or after the method of determining its potency. In some embodiments, the immune cell is genetically altered to respond to a specified antigen. The antigen can be a tumor-specific antigen, for example. In some aspects, the immunotherapy methods include genetically altering the immune cells to present a chimeric antigen receptor (either before or after determining the potency of the immune cell).

As noted throughout the method of determining the potency of an immune cell can be used as part of an adoptive cell transfer treatment. The potent immune cell can be delivered to a subject using a therapeutically acceptable carrier. Intravenous delivery is conventionally used to deliver immunotherapeutic cells, but other methods can also be considered (direct transplant to a localized area of the body in need of immunotherapy, for example).

The therapeutically effective amount can be determined by comparing the amount of cytokine produced by the immune cell to the cytokine potency level required for use of the immune cell in immunotherapy. It is understood and herein contemplated that the therapeutically effective amount depends on the immune cell being administered, the subject being treated, and the disease, disorder, and/or condition being treated. Those of skill in the art will know the appropriate dosage of immune cells to use that will be therapeutically effective for the subject being treated.

A therapeutically effective amount of a potent immune cell encompasses a plurality of potent immune cells. For example, after selecting at least one potent immune cell, the selected cell can be expanded in vitro to produce a plurality of potent immune cells.

The subject receiving the potent immune cells can be any subject that would benefit from immunotherapy (such as for example a subject with an autoimmune disease, inflammatory diseases or disorders, viral diseases and/or bacterial infections). In some embodiments, the subject can be a cancer patient. In some embodiments, the subject can be an individual at high risk of developing cancer, diagnosed with cancer, being treated for cancer, or recovering from cancer after surgery. In some embodiments, the potent immune cells can be delivered to a subject as a prophylactic agent for preventing, inhibiting, or delaying the onset of cancer or a metastasis.

### Methods of Treating a Disease

It is understood and herein contemplated that the potent immune cells identified herein can be used in the treatment of any disease or disorder where adoptive immunotherapy could be used for treatment including, but not limited to autoimmune disease, inflammatory diseases or disorders, viral diseases and/or bacterial infections. Thus, in one aspect, disclosed herein are methods of treating, inhibiting, reducing, preventing, and/or ameliorating a cancer and/or metastasis in a subject comprising a) obtaining one or more immune cells (such as, for example, a T-cell, a macrophage, a NK cell, NK T cell, CAR T cell, and/or CAR NK cell) obtained from an allogeneic or autologous donor); b) contacting an immune cell with an effective amount of a plasma membrane particle, liposome, or an exosome (including engineered exosomes); c) detecting the amount of a cytokine (such as, for example, IL-2, IL-6, IFN-γ, TNF-α, BAFF/TNFSF13B, CD163, CD30/TNFRSF8, Chitinase 3-like 1, gp130, IFN-α2, IL-6Rα, IL-8, IL-10, IL-11, IL-12(p40), IL-12(p70), IL-20, IL-22, IL-26, IL-29/IFN-11, IL-32, IL-34, IL-35, MMP-1, Osteocalcin, OPN, Pentraxin-3, TNF-R1, TNF-R2, TSLP, GM-CSF, MIP-1α, MIP-1β, RANTES, and/or TWEAK/TNFSF12) produced by the immune cell; d) selecting at least one potent immune cell based on the amount of cytokine detected; and e) administering to the subject a therapeutically effective amount of the potent immune cell. In some aspect, the method can further comprise extracting the immune cell from an autologous or allogeneic donor.

It is understood and herein contemplated that it is helpful to be able to administer large numbers of immune cells during immunotherapy, in some embodiments the immune cells are expanded immune cells. Expanded immune cells are those that are grown ex-vivo in order to grow a large number of immune cells. Accordingly, disclosed herein are methods of treating, inhibiting, reducing, preventing, and/or ameliorating an autoimmune disease, inflammatory disease or disorder, viral disease, bacterial infection, cancer and/or metastasis further comprising expanding the at least one potent immune cell prior to delivering a therapeutically effective amount of the at least one potent immune cell.

It is understood and herein contemplated that the disclosed methods of treatment can be used to treat any disease or condition where uncontrolled cellular proliferation occurs including, but not limited to cancer and metastasis. A representative but non-limiting list of cancers that the disclosed methods of using potent immune cells can be used to treat is the following: lymphoma, B cell lymphoma, T cell lymphoma, mycosis fungoides, Hodgkin's Disease, myeloid leukemia, bladder cancer, brain cancer, nervous system cancer, head and neck cancer, squamous cell carcinoma of head and neck, lung cancers such as small cell lung cancer and non-small cell lung cancer, neuroblastoma/glioblastoma, ovarian cancer, skin cancer, liver cancer, melanoma, squamous cell carcinomas of the mouth, throat, larynx, and lung, cervical cancer, cervical carcinoma, breast cancer, and epithelial cancer, renal cancer, genitourinary cancer, pulmonary cancer, esophageal carcinoma, head and neck carcinoma, large bowel cancer, hematopoietic cancers; testicular cancer; colon cancer, rectal cancer, prostatic cancer, or pancreatic cancer.

Examples of autoimmune diseases that can be treated using the disclosed methods include, but are not limited to Achalasia, Acute disseminated encephalomyelitis, Acute motor axonal neuropathy, Addison's disease, Adiposis dolorosa , Adult Still's disease, Agammaglobulinemia, Alopecia areata, Alzheimer's disease, Amyloidosis, Ankylosing spondylitis, Anti-GBM/Anti-TBM nephritis, Antiphospholipid syndrome, Aplastic anemia , Autoimmune angioedema, Autoimmune dysautonomia, Autoimmune encephalomyelitis, Autoimmune enteropathy, Autoimmune hemolytic anemia, Autoimmune hepatitis, Autoimmune inner ear disease (AIED), Autoimmune myocarditis, Autoimmune oophoritis, Autoimmune orchitis, Autoimmune pancreatitis, Autoimmune polyendocrine syndrome , Autoimmune retinopathy, Autoimmune urticaria, Axonal & neuronal neuropathy (AMAN), Baló disease, Behcet's disease, Benign mucosal emphigoid, Bickerstaffs encephalitis , Bullous pemphigoid, Castleman disease (CD), Celiac disease, Chagas disease, Chronic fatigue syndrome, Chronic inflammatory demyelinating polyneuropathy (CIDP), Chronic recurrent multifocal osteomyelitis (CRMO), Churg-Strauss Syndrome (CSS), Eosinophilic Granulomatosis (EGPA), Cicatricial pemphigoid, Cogan's syndrome, Cold agglutinin disease, Congenital heart block, Coxsackie myocarditis, CREST syndrome, Crohn's disease, Dermatitis herpetiformis, Dermatomyositis, Devic's disease (neuromyelitis optica), Diabetes mellitus type 1, Discoid lupus, Dressler's syndrome, Endometriosis, Enthesitis, Eosinophilic esophagitis (EoE), Eosinophilic fasciitis, Erythema nodosum, Essential mixed cryoglobulinemia, Evans syndrome, Felty syndrome, Fibromyalgia, Fibrosing alveolitis, Giant cell arteritis (temporal arteritis), Giant cell myocarditis, Glomerulonephritis, Goodpasture's syndrome, Granulomatosis with Polyangiitis, Graves' disease, Guillain-Barre syndrome, Hashimoto's encephalopathy, Hashimoto's thyroiditis, Hemolytic anemia, Henoch-Schonlein purpura (HSP), Herpes gestationis or pemphigoid gestationis (PG), Hidradenitis Suppurativa (HS) (Acne Inversa), Hypogammalglobulinemia, IgA Nephropathy, IgG4-related sclerosing disease, Immune thrombocytopenic purpura (ITP), Inclusion body myositis (IBM), Interstitial cystitis (IC), Inflamatory Bowel Disease (IBD), Juvenile arthritis, Juvenile diabetes (Type 1 diabetes), Juvenile myositis (JM), Kawasaki disease, Lambert-Eaton syndrome, Leukocytoclastic vasculitis, Lichen planus, Lichen sclerosus, Ligneous conjunctivitis, Linear IgA disease (LAD), Lupus nephritis, Lupus vasculitis, Lyme disease chronic, Meniere's disease, Microscopic polyangiitis (MPA), Mixed connective tissue disease (MCTD), Mooren's ulcer, Mucha-Habermann disease, Multifocal Motor Neuropathy (MMN) or MMNCB, Multiple sclerosis, Myasthenia gravis, Myositis, Narcolepsy, Neonatal Lupus, Neuromyelitis optica, Neutropenia, Ocular cicatricial pemphigoid, Optic neuritis, Ord's thyroiditis, Palindromic rheumatism (PR), PANDAS, Paraneoplastic cerebellar degeneration (PCD), Paroxysmal nocturnal hemoglobinuria (PNH), Parry Romberg syndrome, Pars planitis (peripheral uveitis), Parsonnage-Turner syndrome, Pemphigus, Peripheral neuropathy, Perivenous encephalomyelitis, Pernicious anemia (PA), POEMS syndrome, Polyarteritis nodosa, Polyglandular syndromes type I, II, III, Polymyalgia rheumatica, Polymyositis, Postmyocardial infarction syndrome, Postpericardiotomy syndrome, Primary biliary cirrhosis, Primary sclerosing cholangitis, Progesterone dermatitis, Psoriasis, Psoriatic arthritis, Pure red cell aplasia (PRCA), Pyoderma gangrenosum, Raynaud's phenomenon, Reactive Arthritis, Reflex sympathetic dystrophy, Relapsing polychondritis, Restless legs syndrome (RLS), Retroperitoneal fibrosis, Rheumatic fever, Rheumatoid arthritis, Rheumatoid vasculitis, Sarcoidosis, Schmidt syndrome, Schnitzler syndrome, Scleritis, Scleroderma, Sjogren's syndrome, Sperm & testicular autoimmunity, Stiff person syndrome (SPS), Subacute bacterial endocarditis (SBE), Susac's syndrome, Sydenham chorea, Sympathetic ophthalmia (SO), Systemic Lupus Erythematosus, Systemic scleroderma, Takayasu's arteritis, Temporal arteritis/Giant cell arteritis, Thrombocytopenic purpura (TTP), Tolosa-Hunt syndrome (THS), Transverse myelitis, Type 1 diabetes, Ulcerative colitis (UC), Undifferentiated connective tissue disease (UCTD), Urticaria, Urticarial vasculitis, Uveitis, Vasculitis, Vitiligo, Vogt-Koyanagi-Harada Disease, and Wegener's granulomatosis (or Granulomatosis with Polyangiitis (GPA)).

The following example is included to demonstrate preferred embodiments of the invention. It should be appreciated by those of skill in the art that the techniques disclosed in the examples, which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### EXAMPLE

The assays disclosed herein intend to test the potency of therapeutic immune cells that would address the problems existing for current standard methods, and satisfy the FDA requirements. To achieve this, K562-derived exosomes are used as a surrogate to induce cytokine production in immune cells. K562 (chronic myeloid leukemia cell line) is widely being used as universal control target cell line in cytotoxicity assays for immune cells. These K562 cells regularly release exosome - multivesicular bodies formed by inward budding of endosomal membranes. The exosomes would induce the cytokine production like K562 cells, but would remove variabilities caused by the use of target tumor cells. The assay would eliminate the need to have a fully operational research laboratory to test the potency of therapeutic immune cells at multiple clinical infusion sites, and would provide a quicker turnaround time for such tests.

### Testing the Potency of Theraneutic Immune Cells Using K562-Derived Exosomes as a Stimulatory Agent

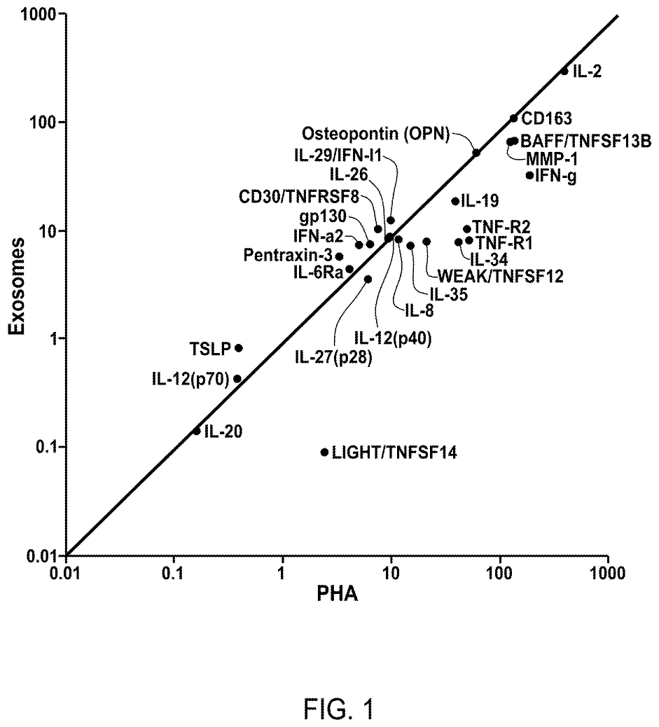

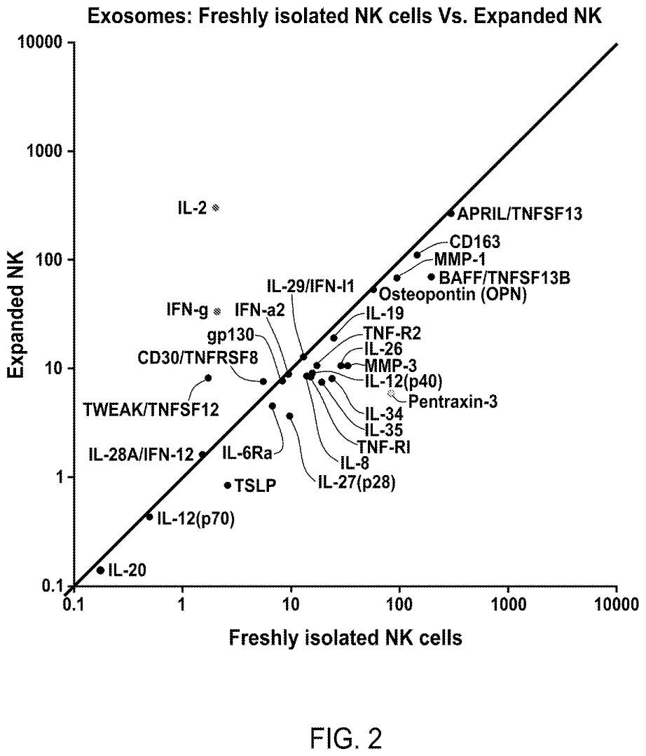

The ability of exosomes to assay immune cell potency is demonstrated in FIGS. 1 and 2. FIG. 1 provides a graph that therapeutic NK cells can produce IL-2 or IFN-γ via either of the potency assay-PHA or exosome-potency assay or exosomes, demonstrating that the exosome potency assay can be used for therapeutic NK cells. FIG. 2 provides a graph showing that the exosome potency assay can also be used to identify expanded therapeutic NK cells via high IL-2 and IFN-g production. Also, freshly isolated NK cells from healthy donor get stimulated by this potency assay and secrete other cytokines such as APRIL/TNSF13, CD163, and BAFF that can be used for diagnostics for NK cell deficiencies in patients.

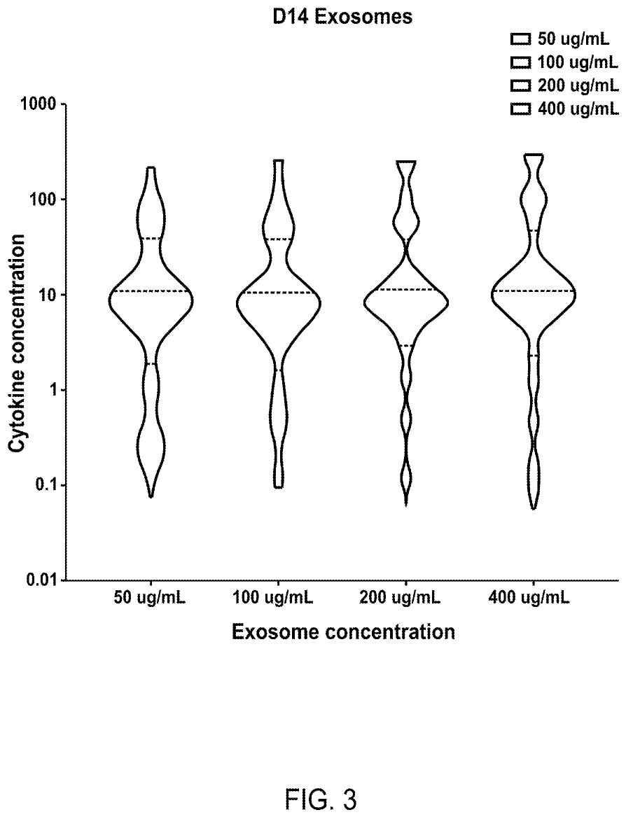

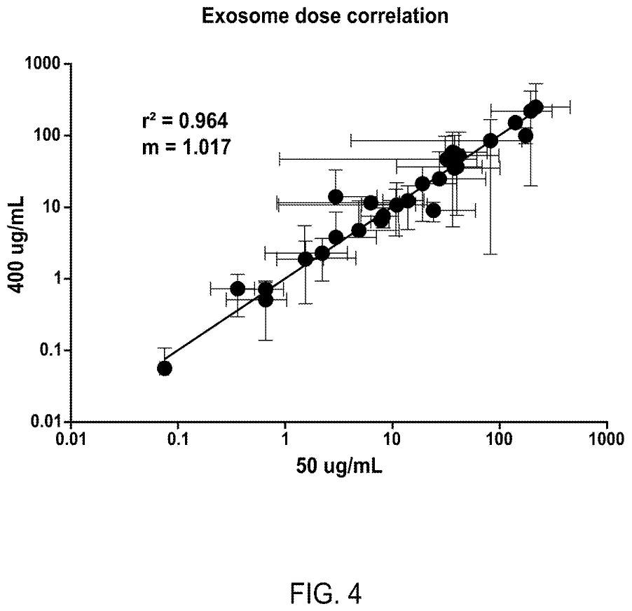

We tested production of 29 different cytokines and chemokines by NK cells from 9 different donors in response to varying concentrations of CSTX002 exosomes. There was no difference in expression patterns between the concentrations (FIG. 3). To specifically assess reproducibility of each cytokine and variation across concentrations of exosomes, tested the correlation of low (50 ug/mL) and high (400 ug/mL) concentrations of exosomes across 29 cytokines and chemokines by NK cells from 9 different donors. There was very high correlation (r2=0.964) and less than 2% variation (slope−1), indicating that a wide range of exosome concentrations will yield identical results (FIG. 4).

Unless defined otherwise, all technical and scientific terms used herein have the same meanings as commonly understood by one of skill in the art to which the disclosed invention belongs. Publications cited herein and the materials for which they are cited are specifically incorporated by reference. However, it should be appreciated that any patent, publication, or other disclosure material, in whole or in part, that is said to be incorporated by reference herein is incorporated herein only to the extent that the incorporated material does not conflict with existing definitions, statements, or other disclosure material set forth in this disclosure. As such, and to the extent necessary, the disclosure as explicitly set forth herein supersedes any conflicting material incorporated herein by reference. Any material, or portion thereof, that is said to be incorporated by reference herein, but which conflicts with existing definitions, statements, or other disclosure material set forth herein will only be incorporated to the extent that no conflict arises between that incorporated material and the existing disclosure material.

Those skilled in the art will recognize, or be able to ascertain using no more than routine experimentation, many equivalents to the specific embodiments of the invention described herein. While the invention has been described with reference to particular embodiments and implementations, it will understood that various changes and additional variations may be made and equivalents may be substituted for elements thereof without departing from the scope of the invention or the inventive concept thereof. In addition, many modifications may be made to adapt a particular situation or device to the teachings of the invention without departing from the essential scope thereof. Such equivalents are intended to be encompassed by the following claims. It is intended that the invention not be limited to the particular implementations disclosed herein, but that the invention will include all implementations falling within the scope of the appended claims.

1. A method of assaying the potency of an immune cell, comprising contacting an immune cell with an effective amount of a plasma membrane particle, a liposome, or an exosome and detecting the amount of a cytokine produced by the immune cell.

2. The method of claim 1, further comprising the step of comparing the amount of cytokine produced to the cytokine potency level required for use of the immune cell in immunotherapy.

3. The method of claim 1, wherein the amount of a plurality of cytokines is determined.

4. The method of claim 1, wherein the immune cell is a T-cell, a macrophage, a Natural Killer (NK) cell, NK T cell, chimeric antigen receptor (CAR) T cell, or CAR NK cell.

5. The method of claim 1, wherein the immune cell is an NK cell.

6. The method of claim 1, wherein the exosome is a cancer cell exosome.

7. The method of claim 1, wherein the amount of cytokine is detected using an immunoassay.

8. The method of claim 1, wherein the cytokine is selected from the group comprising interleukin (IL)-2 (IL-2), IL-6, interferon (IFN)-γ (IFN-γ), B cell activating factor/tumor necrosis factor (TNF) ligand superfamily member 13B (BAFF/TNFSF13B), TNF-α, cluster of differentiation (CD) 163 (CD163), CD30/TNFRSF8, Chitinase 3-like 1, gp130, IFN-a2, IL-6Ra, IL-8, IL-10, IL-11, IL-12(p40), IL-12(p70), IL-20, IL-22, IL-26, IL-29/IFN-11, IL-32, IL-34, IL-35, matrix metalloproteinase-1 (MMP-1), Osteocalcin, Osteopontin (OPN), Pentraxin-3, tumor necrosis factor (TNF)-receptor 1 (TNF-R1), TNF-R2, thymic stromal lymphopoetin (TSLP), granulocyte-macrophage colony-stimulating factor (GM-CSF), leukemia inhibitory factor (LIF), and the chemokines macrophage inflammatory protein (MIP)-1α (MIP-1α), RANTES, and/or TNF-related weak inducer of apoptosis (TWEAK)/TNF superfamily member 12 (TWEAK/TNFSF12).

9. The method of claim 1, wherein the immune cell is contacted with an effective amount of the plasma membrane particle, the liposome, or the exosome for at least 4 hours.

10. The method of claim 1, wherein the plasma membrane particle, the liposome, or the exosome is provided at a concentration of 50 μg/mL to 400 μg/mL.

11. A kit for assaying the potency of an immune cell, comprising a container including an effective amount of a plasma membrane particle and/or an exo some and a buffer suitable for immune cells.

12. The kit of claim 11, wherein the plasma membrane particle, the liposome, or the exosome is provided at a concentration of 50 μg/mL to 400 μg/mL.

13. The kit of claim 11, wherein the container is an Eppendorf microcentrifuge tube.

14. The kit of claim 11, wherein the kit further comprises instructions for using the kit to stimulate cytokine production by an immune cell.

15. An immunotherapy method comprising;
a. performing the method of claim 1 on multiple immune cells to determine the potency of each immune cell;
b. selecting at least one potent immune cell based on the amount of cytokine detected; and
c. administering a therapeutically effective amount of the potent immune cell to a subject in need thereof as an immunotherapeutic.

16. The immunotherapy method of claim 15, further comprising extracting the multiple immune cells from an allogeneic or autologous donor prior to assaying the potency of the immune cell.

17. The immunotherapy method of claim 15, further comprising expanding the at least one potent immune cell prior to delivering a therapeutically effective amount of the potent immune cell.

18. The immunotherapy method of claim 15, further comprising directing the multiple immune cells or the potent immune cell to respond to a specified antigen.

19. The immunotherapy method of claim 18, further comprising genetically altering the multiple immune cells or the potent immune cell to present a chimeric antigen receptor.

20. A method of treating, inhibiting, reducing, preventing, and/or ameliorating a cancer and/or metastasis in a subject comprising:
a. obtaining one or more immune cells;
b. contacting an immune cell with an effective amount of a plasma membrane particle, a liposome, or an exosome;
c. detecting the amount of a cytokine produced by the immune cell;
d. selecting at least one potent immune cell based on the amount of cytokine detected; and
e. administering to the subject a therapeutically effective amount of the potent immune cell.

21. The method of treating, inhibiting, reducing, preventing, and/or ameliorating a cancer and/or metastasis in a subject of claim 20, wherein the one or more immune cells is obtained from an allogeneic or autologous donor.

22. The method of treating, inhibiting, reducing, preventing, and/or ameliorating a cancer and/or metastasis in a subject of claim 20, further comprising extracting the multiple immune cells from an allogeneic or autologous donor.

23. The method of treating, inhibiting, reducing, preventing, and/or ameliorating a cancer and/or metastasis in a subject of claim 20, wherein the immune cell is a T-cell, a macrophage, a Natural Killer (NK) cell, NK T cell, chimeric antigen receptor (CAR) T cell, or CAR NK cell.

24. The method of treating, inhibiting, reducing, preventing, and/or ameliorating a cancer and/or metastasis in a subject of claim 20, further comprising expanding the at least one potent immune cell prior to delivering a therapeutically effective amount of the at least one potent immune cell.
